(19) United States
(12) Patent Application Publication
(10) Pub. No.: US 2011/0257431 A1
(43) Pub. Date: Oct. 20, 2011

(54) PROCESS FOR PRODUCING SIDE PRODUCT-FREE AMINOCARBOXYLATES

(75) Inventors: Robert Baumann, Mannheim (DE); Markus Christian Biel, Mannheim (DE); Gordon Brasche, Frankfurt (DE); Axel Franzke, Mannheim (DE); Thomas Heidenfelder, Randolph, NJ (US); Paul Klingelhoefer, Mannheim (DE); Alfred Oftring, Bad Durkheim (DE); Marie Katrin Schroter, Dannstadt-Schauernheim (DE)

(73) Assignee: BASF SE, Ludwigshafen (DE)

(21) Appl. No.: 13/050,495

(22) Filed: Mar. 17, 2011

Related U.S. Application Data
(60) Provisional application No. 61/315,107, filed on Mar. 18, 2010.

(51) Int. Cl. C07C 227/02

(52) U.S. Cl. 562/526

(57) ABSTRACT

The present invention relates to a process for preparing aminocarboxylates, proceeding from amines, by employing a reaction sequence composed of ethoxylation to amino alcohols and subsequent oxidative dehydrogenation to the corresponding aminocarboxylates, especially the alkali metal or alkaline earth metal salts of the complexing agents MGDA (methylglycinediacetic acid), EDTA (ethylenediaminetetra-acetic acid) and GLDA (glutamic acid diacetic acid) or the free acids thereof.

The present invention relates to a process for preparing aminocarboxylates proceeding from the amines 1 and 4 by employing a reaction sequence composed of ethoxylation to the amino alcohols 2 and 5 and subsequent oxidative dehydrogenation to the corresponding aminocarboxylates 3 and 6 (for example the alkali metal or alkaline earth metal salts of the complexing agents MGDA (methylglycinediacetic acid), EDTA (ethylenediaminetetraacetic acid) or GLDA (glutamic acid diacetic acid) or the free acids thereof).

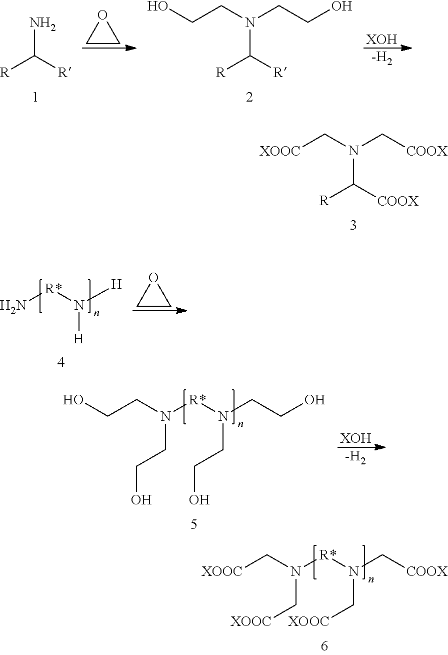

R=alkyl, alkenyl, alkynyl, aryl, aralkyl, alkylenecarboxyl, hydroxyalkyl, hydroxyaralkyl, alkylenesulfonate

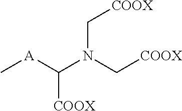

where A=C1 to C12 alkylene bridge or a chemical bond

### R′=COOX, CH₂OH

R*=alkylene
X=alkali metals or alkaline earth metals, preferably sodium and potassium

### N=1−10

The ethoxylation of amines is performed on the industrial scale, typically at temperatures greater than 120° C. For instance, ethanolamines are prepared proceeding from ammonia (solution of 20 to 30% by mass in water) and ethylene oxide at temperatures around 150° C. and at pressures of 30 to 150 bar (H.-J. Arpe, Industrielle Organische Chemie [Industrial Organic Chemistry]). N-Alkylethanolamines are even prepared at temperatures up to 170° C. (Ullmann's Encyclopedia). WO 98/38153 describes the ethoxylation of ethylenediamine in isopropanol as a solvent with 4 equivalents of ethylene oxide at standard pressure and a reaction temperature of 140 to 180° C. The corresponding ethoxylation in pure substance is described in U.S. Pat. No. 3,907,745, at somewhat lower temperatures of 120 to 130° C.

The oxidative dehydrogenation of amino alcohols with alkali metal hydroxides is typically performed under pressure and at temperatures of 140 to 220° C. using copper catalysts. The catalysts consist, for example, of doped or undoped Raney Cu (described, for example, in EP 1 125 633, EP 1 125 634, WO 04/24091, WO 00/066539, EP 1 067 114, WO 00/032310). The dopants used are generally one or more metals, for example Pt, Fe, Cr (EP 1 125 633, EP 1 125 634) Cr, Mo, V, Bi, Sn, Sb, Pb, Ge (WO 04/24091) or Ag (EP 1 067 114).

In other examples, Cu is applied to alkali-stable supports directly or via anchor metals (e.g. Os, Ir, Rh, Pt, Pd) (e.g. WO 01/77054, WO 03/022140, WO 98/50150). Precipitated Cu catalysts with further metal oxides have also been described (e.g. WO 03/051513 (Cu, Fe), EP 0 506 973, WO 98/13140 (Cu, Zr, Ca)). There have also been isolated reports about conversion over noble metal systems (e.g. EP 0 201 957).

A problem in the preparation especially of complexing agents such as MGDA (methylglycinediacetic acid), EDTA (ethylenediaminetetraacetic acid) or GLDA (glutamic acid diacetic acid) and salts thereof is that relatively high contents of by-products are obtained in a simple performance of the two process steps. In order to keep the content of such by-products in the end product low, expensive operations, which are complex in terms of apparatus, to purify the end product and/or the intermediate are required.

It is therefore an object of the present invention to provide a process which does not have the disadvantage mentioned, i.e. which affords an end product with a low by-product content and in which operations to purify the end product and/or intermediate are dispensable.

This object is surprisingly achieved by the process according to claims 1 to 10:

According to the invention, the object is achieved by a process for preparing aminocarboxylates, in which, in a first stage, an amine is ethoxylated at a reaction temperature in the range from 30 to 100° C. to give an alkanolamine, and the alkanolamine thus formed is dehydrogenated in a second stage oxidatively to give an aminocarboxylate, where the salts which form can also be converted to the corresponding aminocarboxylic acids.

Preference is given to a process in which the amine is selected from the group of the amines of the formula 1 or 4

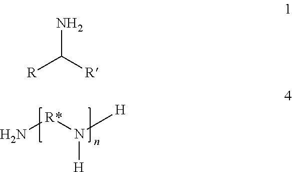

where

R is an alkyl, alkenyl, alkynyl, aryl, aralkyl, alkylenecarboxyl, hydroxyalkyl, hydroxy-aralkyl, alkylenesulfonate or a substituent

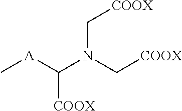

where A=C1 to C12 alkylene bridge, or a chemical bond

### R′ is COOX or CH2OH,

R* is an alkylene radical,
X is an alkali metal or alkaline earth metal and
n is from 1 to 10.

R is more preferably relatively long alkyl or alkenyl radicals of C1 to C30 alkyl and C2 to C30 alkenyl, alkylenecarboxylates or else alkylenesulfonates, hydroxyalkyl or hydroxyaryl groups and double alkylglycinediacetic acids such as diaminosuccinic acid (A=“chemical bond”) or diaminopimelic acid (A=—(CH₂)3—) with

### R=

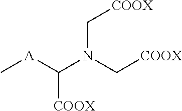

where A=a C1 to C12 alkylene bridge or a chemical bond.

Particular preference is given to a process in which the amine is selected from the group consisting of alanine, glutamic acid and salts thereof, and ethylenediamine.

With regard to the process parameters, there are preferred embodiments. Preference is thus given to a process in which the reaction temperature in the first stage is in the range from 40 to 90° C., preferably in the range from 60 to 80° C.

With regard to the temperature profile too, there are preferred variants. Preference is thus given to a process in which the reaction temperature in the first stage varies by less than 60° C., preferably by less than 40° C., over the reaction time.

The performance of the process as a batchwise, semibatchwise or continuous process is preferred. A process in which (at least) one reactor selected from the group consisting of stirred tank reactor, loop reactor and tubular reactor is used is particularly preferred.

This is possible using various reactor models such as stirred tank reactors of various designs, loop reactors (gas circulation reactor, plunging jet reactor, jet nozzle reactor or high-loading packed column) or tubular reactors (gas phase-free or with gas phase).

A process in which the reactor consists essentially of a material with a thermal conductivity coefficient greater than 5 W/K*m is particularly suitable. “Essentially” means that more than 50%, preferably more than 80% and more preferably more than 90% of the reactor material consists of a material with a corresponding thermal conductivity coefficient.

Particularly suitable materials for this purpose are found to include 1.4541 (V2A steel), 1.4571 (V4A steel), 2.4610 (HC4) with a thermal conductivity coefficient greater than 5 W/K*m, in order to enable efficient removal of heat in the industrial process.

Equally preferred is a process in which the solvent of the first stage is selected from protic solvents such as water, alcohols, preferably short-chain alcohols, and especially methanol, ethanol, 2-propanol and/or polar aprotic solvents such as dimethyl sulfoxide, dimethylformamide or N-methylpyrrolidone.

A process in which the alkanolamine formed in the first stage is dehydrogenated directly constitutes a further preferred embodiment. Direct dehydrogenation means that preference is given to those processes in which there is no apparatus for removing substances with boiling points greater than 200° C. (at standard pressure), on the basis of different boiling points, between the first and second stages. This is simpler in apparatus terms and hence saves one process step with comparably good end product quality.

Particular preference is given to a process in which the end product too is not purified further, but is used directly in the corresponding applications, for example as an additive for industrial cleaning formulations for hard surfaces of metal, plastic, coating material or glass, in alkaline cleaning formulations for the drinks and foods industry, especially for bottle cleaning in the drinks industry and in apparatus cleaning in dairies, in breweries, in the preserves industry, in the bakery industry, in the sugar industry, in the fat-processing industry and in the meat-processing industry, in dishware cleaning formulations, especially in phosphate-free compositions for machine dishwashing in machine dishwashers in the household or in commercial premises, for example large kitchens or restaurants, in bleaching baths in the paper industry, in photographic bleaching and bleach fixing baths, in pretreatment and bleaching in the textile industry, in electrolytic baths for masking of contaminating heavy metal cations, and also in the field of plant foods for remedying heavy metal deficits as copper, iron, manganese and zinc complexes. In principle, use is advantageous anywhere where precipitations of calcium, magnesium or heavy metal salts disrupt industrial processes and should be prevented (prevention of deposits and encrustations in tanks, pipelines, spray nozzles or generally on smooth surfaces), and also for stabilization of phosphates in alkaline degreasing baths and prevention of the precipitation of lime soaps. in order thus to prevent the tarnishing of non-iron surfaces and to prolong the service life of alkaline cleaning baths. In addition, they find use in pulverulent or liquid detergent formulations for textile washing as builders and preservatives. In soaps, they prevent metal-catalyzed oxidative decompositions, and also in pharmaceuticals, cosmetics and foods.

The dehydrogenation is effected with the aid of a base from the group of the alkali metal and alkaline earth metal hydroxides, preferably NaOH or KOH, particular preference being given to NaOH. The temperature of the second stage is typically in the range from 140 to 240° C., preferably in the range from 150 to 210° C. and more preferably in the range from 160 to 200° C. The pressure is typically in the range from standard pressure to 100 bar, preferably from 5 to 50 bar and more preferably in the range from 8 to 20 bar and even more preferably from 10 to 20 bar.

A process in which the dehydrogenation is performed with a catalyst, the main and secondary constituents of which is/are selected from groups 4 to 12 of the Periodic Table, is particularly preferred; very particular preference is given to a process in which the dehydrogenation is performed with a catalyst which comprises (at least) one metal which is selected from the group consisting of: Cu, Fe, Co, Ni, Zr, Hf, Ag, Pd and Pt. The catalyst can be used, for example, in the form of powder or shaped bodies (e.g. extrudates, tablets etc.), or in the form of an unsupported catalyst or supported catalyst, and may consist of metals and metal oxides.

A process in which the NTA content in the direct product of the second stage is less than 1% by mass, based on the main product, forms a further part of the subject matter of the present invention.

In addition to the salts (aminocarboxylates) themselves, the corresponding amino-carboxylic acids are also obtainable after acidification. The direct product of the second stage is understood to mean the reaction discharge as obtained in the oxidative dehydrogenation. Thereafter, in the case of a suspension method, the catalyst can be sedimented and filtered off. In addition, a desired water content can subsequently be established or a bleaching can be carried out, for example with hydrogen peroxide or UV light.

The present invention is illustrated in detail hereinafter by nonlimiting examples:

### EXAMPLE 1

3.743 kg (20.00 mol) of glutamic acid monosodium salt monohydrate are suspended in 5.599 kg of water and admixed with 1.578 kg (20.00 mol) of 50.7% by mass sodium hydroxide solution. The resulting mixture was charged into a 20 l autoclave (2.4610 material) and, after appropriate inertization, nitrogen was injected to 20 bar. Subsequently, 2.026 kg (46.00 mol) of ethylene oxide were metered in at 40-45° C. within 8 h, and the mixture was stirred at this temperature for a further 2 h. After the removal of the unconverted residues of ethylene oxide, the autoclave was emptied. In this way, 12.862 kg of aqueous reaction discharge were obtained as a clear, colorless, viscous solution.

418 g (0.650 mol based on glutamic acid monosodium salt monohydrate) of this crude product were initially charged with 53.0 g (1.33 mol) of sodium hydroxide powder, 12.7 g of water and 7.5 g of a copper-iron catalyst prepared according to WO 03/051513 in a 1.2 l autoclave (2.4610 material). The reactor was closed, nitrogen was injected to 5 bar, and the reactor was then heated to 190° C. The temperature was held for 6 h. The stirrer speed over the entire experimental duration was 700 rpm. The hydrogen which formed was removed continuously through a 15 bar pressure-regulating valve. After the end of the experiment, the reactor was purged with nitrogen at room temperature and then emptied. The product was obtained as a clear, colorless, viscous solution. By iron binding capacity, a glutamic acid-N,N-diacetic acid tetrasodium salt (GLDA-Na₄) content of 42.2% by mass was determined, which corresponds to a yield of 88.6% of theory based on glutamic acid monosodium salt monohydrate used.

### EXAMPLE 2

4.365 kg (49.00 mol) of alanine were suspended in 2.600 kg of water and admixed with 3.920 kg (49.00 mol) of 50% by mass sodium hydroxide solution. The resulting mixture was charged into a 20 l autoclave (2.4610 material) and, after appropriate inertization, nitrogen was injected to 20 bar. Subsequently, 4.749 kg (107.8 mol) of ethylene oxide were metered in at 40-45° C. within 8 h, and the mixture was stirred at this temperature for a further 2 h. After the removal of the unconverted residues of ethylene oxide, the autoclave was emptied. In this way, 15.597 kg of aqueous reaction discharge were obtained as a clear, colorless, viscous solution.

328 g (1.03 mol based on alanine) of this crude produce were initially charged with 197 g (2.46 mol) of 50% by mass sodium hydroxide solution, 18 g of water and 45 g of Raney copper (from Evonik Degussa GmbH) in a 1.7 l autoclave (2.4610 material). The reactor was closed, nitrogen was injected to 5 bar, and the reactor was then heated to 190° C. within 2.25 h. This temperature was held for 16 h. The stirrer speed over the entire experimental duration was 500 rpm. The hydrogen which formed was removed continuously through a 10 bar pressure-regulating valve. After the end of the experiment, the reactor was purged with nitrogen at room temperature, the reaction discharge was diluted with 484 g of water and the reactor was then emptied. The product was obtained as a clear, colorless, viscous solution. By means of HPLC, a yield of methylglycine-N,N-diacetic acid trisodium salt (MGDA-Na₃) of 92.0% of theory based on alanine used was determined.

### EXAMPLE 3

178 g (2.00 mol) of alanine were suspended in 106 g of water and admixed with 160 g (2.00 mol) of 50% by mass sodium hydroxide solution. The resulting mixture was charged into a 2.5 l autoclave (1.4571 material) and, after appropriate inertization, nitrogen was injected to 1 bar. Subsequently, 189 g (4.30 mol) of ethylene oxide were metered in at 80-89° C. within 2 h, and the mixture was stirred at this temperature for a further 3 h. After the removal of the unconverted residues of ethylene oxide, the autoclave was emptied. In this way, 624 g of aqueous reaction discharge were obtained as a clear, colorless, viscous solution.

328 g (1.05 mol based on alanine) of this crude produce were initially charged with 208 g (2.60 mol) of 50% by mass sodium hydroxide solution, 39 g of water and 45 g of Raney copper (from Evonik Degussa GmbH) in a 1.7 l autoclave (2.4610 material). The reactor was closed, nitrogen was injected to 5 bar, and the reactor was then heated to 190° C. within 2.25 h. This temperature was held for 16 h. The stirrer speed over the entire experimental duration was 500 rpm. The hydrogen which formed was removed continuously through a 10 bar pressure-regulating valve. After the end of the experiment, the reactor was purged with nitrogen at room temperature, the reaction discharge was diluted with 403 g of water and the reactor was then emptied. The product was obtained as a clear, colorless, viscous solution. By means of HPLC, a yield of methylglycine-N,N-diacetic acid trisodium salt (MGDA-Na₃) of 91.3% of theory based on alanine used was determined.

### COMPARATIVE EXAMPLE

267 g (3.00 mol) of alanine were suspended in 159 g of water and admixed with 240 g (3.00 mol) of 50% by mass sodium hydroxide solution. The resulting mixture was charged into a 2.5 l autoclave (1.4571 material) and, after appropriate inertization, nitrogen was injected to 20 bar. Subsequently, 291 g (6.60 mol) of ethylene oxide were metered in at 140-145° C. within 5 h, and the mixture was stirred at this temperature for a further 2 h. After the removal of the unconverted residues of ethylene oxide, the autoclave was emptied. In this way, 930 g of aqueous reaction discharge were obtained as a clear, yellowish, viscous solution.

322 g (1.04 mol based on alanine) of this crude produce were initially charged with 208 g (2.60 mol) of 50% by mass sodium hydroxide solution, 40 g of water and 45 g of

Raney copper (from Evonik Degussa GmbH) in a 1.7 l autoclave (2.4610 material). The reactor was closed, nitrogen was injected to 5 bar, and the reactor was then heated to 190° C. within 2.25 h. This temperature was held for 16 h. The stirrer speed over the entire experimental duration was 500 rpm. The hydrogen which formed was removed continuously through a 10 bar pressure-regulating valve. After the end of the experiment, the reactor was purged with nitrogen at room temperature, the reaction discharge was diluted with 424 g of water and the reactor was then emptied. The product was obtained as a clear, colorless, viscous solution. By HPLC, in spite of full conversion, a yield of methylglycine-N,N-diacetic acid trisodium salt (MGDA-Na₃) of only 74.4% of theory based on alanine used was determined.

1. A process for preparing aminocarboxylates, in which, in a first stage, an amine is ethoxylated at a reaction temperature in the range from 30 to 100° C. to give an alkanolamine, and the alkanolamine thus formed is dehydrogenated in a second stage oxidatively to give an aminocarboxylate.

2. The process according to claim 1, in which the amine is selected from the group consisting of alanine, glutamic acid and salts thereof, and ethylenediamine.

3. The process according to either of claims 1 and 2, in which the reaction temperature in the first stage varies by less than 60° C. over the reaction time.

4. The process according to any of claims 1 to 3, which is performed as a batchwise, semibatchwise or continuous process.

5. The process according to any of claims 1 to 4, in which a reactor selected from the group consisting of stirred tank reactor, loop reactor and tubular reactor is used.

6. The process according to any of claims 1 to 5, in which the reactor consists essentially of a material with a thermal conductivity coefficient greater than 5 W/K*m.

7. The process according to any of claims 1 to 6, in which the solvent of the first stage is selected from protic and/or polar aprotic solvents.

8. The process according to any of claims 1 to 7, in which the alkanolamine formed in the first stage is dehydrogenated directly.

9. The process according to any of claims 1 to 8, in which the dehydrogenation is performed with a catalyst which comprises a metal which is selected from the group consisting of: Cu, Fe, Co, Ni, Zr, Hf, Ag, Pd and Pt.

10. The process according to any of claims 1 to 9, in which the NTA content in the direct product of the second stage is less than 1% by mass, based on the main product.
